(19) United States
(12) Patent Application Publication
(10) Pub. No.: US 2006/0280707 A1
(43) Pub. Date: Dec. 14, 2006

(54) Composition and manufacturing method of self-diagnosis and reduction catalyst processing for permanent & straighter

(76) Inventors: Jong-ho Park, Seoul (KR); Chang-koo Shim, Seoul (KR); Sang-Uk Chung, Incheon (KR)

Correspondence Address: Ronald R Santucci;Frommer Lawrence & Haug, 745 Fifth Avenue, New York, NY, 10151, US

(21) Appl. No.: 10/544,912

(22) PCT Filed: Apr. 23, 2003
(86) PCT No.: PCT/KR03/00823

§ 371 Date: Aug. 8, 2006

(30) Foreign Application Priority Data
Feb. 10, 2003 (KR) 10 2003-0008234

(51) Int. Cl. A61K 8/00

(52) U.S. Cl. 424/70.2

(57) ABSTRACT

The present invention relates to compositions of first agents of a permanent hair waving agent and a hair straightener for self-diagnosis with a reducing catalyst, respectively, and their manufacturing methods thereof. More specifically, the present invention relates to compositions of first agents a permanent hair waving agent and a hair straightener for self-diagnosis with a reducing catalyst, respectively, and their manufacturing methods thereof, wherein each of the first agents is a reducing agent prepared in two different solutions of A and B and mixed in a predetermined ratio immediately prior to use. Upon mixing, the first agent gives rise to a peculiar color thus enabling to identify both active ingredients and the properties of the solutions being used well in advance of hair styling performance -+y assisting proper selection of a permanent hair waving agent and a hair ,˜ner most suitable for each hair kind thus securing safe performance of hair a also preventing hair damage.

### TECHNICAL FIELD

The present invention relates to a first agent for a permanent hair waving agent and a hair straightener for self-diagnosis with a reducing catalyst and its preparing method, particularly, to a first agent for a permanent hair waving agent and a hair straightener prepared by mixing the solutions divided into A and B in a predetermined ratio for use. The present first agent takes on a unique color, thus enabling to identify the presence of its main ingredient and property (pH) prior to performance. Therefore, any user can select a suitable first agent according to the quality and condition of his hair. Consequently, the first agent according to this invention ensures prevention of hair damage, secures safe performance of hair styling of elastic wave and straightened hair.

### BACKGROUND ART

A permanent hair waving agent and a straightener used for providing wave and straight configuration to hair comprises (a) a first agent (a reducing agent) containing thioglycolic acid or its salts and esters; and cysteine or its salts or acetyl derivatives that allows the reduction of disulfide cystine bonds in hair, (b) a second agent (an oxidizing agent) containing hydrogen peroxide, sodium bromic acid that allows the formation of wave and straight configuration of hair by oxidization-reform of disulfide cystine bonds. Furthermore, for improving its efficiency and applicability, the agent further contains an alkali agent, a penetrant, a stabilizing agent, a wetting agent, a hair tonic, a colorant, an emulsifier and a perfume (see FIGS. 1, 2 and 3).

Depending on main ingredients of the reducing agent in the first agent and its application patter, the first agent is classified into: (a) cold 2 bath type permanent hair waving agent containing thioglycolic acid and its salt as its main ingredients, (b) cold 2 bath type permanent hair waving agent containing cysteine as its main ingredient, (c) cold 2 bath type for curly hair correction use containing thioglycolic acid and its salt as its main ingredients, (d) heating 2 bath type permanent hair waving agent containing thioglycolic acid and its salt as its main ingredients, (e) cold 1 bath type permanent hair waving agent thioglycolic acid and its salt as its main ingredients, (f) exothermic 2 bath type permanent hair waving agent thioglycolic acid and its salt as its main ingredients, and (g) heating 2 bath type for curly hair correction containing thioglycolic acid and its salt as its main ingredients. In addition, for securing safety, the Article 9 of the Korean Cosmetic Law stipulates the criteria for permanent wave products as shown in Table 1.

TABLE 1

| Category | a | b | c | d | e | f | g |
|---|---|---|---|---|---|---|---|
| Contents | — | — | — | — | — | — | — |
| pH | 4.5-9.6 | 8.0-9.5 | 4.5-9.6 | 4.5-9.3 | 9.4-9.6 | 4.5-9.5 | 4.5-9.3 |
| Alkali | Below | Below | Below | Below | 3.5-4.6 ml | Below | Below |
| | 7.0 ml | 12.0 ml | 7.0 ml | 5.0 ml | | 10.0 ml | 5.0 ml |
| Reducing agent | 2.0-7.0% | — | 2.0-7.0% | 1.0-5.0% | 3.0-3.3% | 8-19% | 1.0-5.0% |
| after boiling in | | | | | | | |
| acid | | | | | | | |
| Reducing | Below | — | Below | Below | Below | Below | Below |
| material except | 0.6 ml | | 0.6 ml | 0.6 ml | 0.6 ml | 0.8 ml | 0.6 ml |
| reducing agent | | | | | | | |
| after boiling in | | | | | | | |
| acid | | | | | | | |
| Reducing agent | Below | — | Below | Below | Below | Below | Below |
| after reduction | 4.0% | | 4.0% | 4.0% | 0.5% | 0.5% | 4.0% |
| Pb | Below | Below | Below | Below | Below | Below | Below |
| | 5 ppm | 5 ppm | 5 ppm | 5 ppm | 5 ppm | 5 ppm | 5 ppm |
| Fe | Below | Below | Below | Below | Below | Below | Below |
| | 2 ppm | 2 ppm | 2 ppm | 2 ppm | 2 ppm | 2 ppm | 2 ppm |
| As | Below | Below | Below | Below | Below | Below | Below |
| | 5 ppm | 5 ppm | 5 ppm | 5 ppm | 5 ppm | 5 ppm | 5 ppm |
| Cysteine | — | 3.0-7.5% | — | — | — | — | — |
| Cystine | — | Below | — | — | — | — | — |
| | | 6.65% | | | | | |

The testing methods for categories in Table 1 are suggested by Appendix 5 of the Announcement 2000-27 of the Korean Food and Drug Administration.

As shown in FIGS. 1-3, a permanent hair waving agent and a straightener reduce and cleave disulfide cystine bonds in hair and then oxidize and reform the bonds, which is responsible for hair damage. Further, it generally contains an alkali agent and a pH modifier thus leading to irritation of scalp. Therefore, for maintaining a minimum safety, the criteria for a permanent hair waving agent and a straightener is provided in the Article 9 of the Korean Cosmetic Law and Appendix 5 of the Announcement 2000-27 of the Korean Food and Drug Administration.

However, it has been recognized that, for alleviation of adverse effects such as scalp irritation, the selection of permanent hair waving agent and straightener with the different pH, main ingredient and direction for use depending on type of hair quality (e.g., oily hair, virgin hair, healthy hair, standard hair, porous hair, damaged hair, fine hair and divided hair is necessary.

The present inventors have made intensive studies to develop a novel first agent for a permanent hair waving agent and a straightener that enables to predetermine hair damage and scalp irritation and to selectively use according to the quality and condition of hair.

As a result, the present inventors have developed a novel first agent prepared by mixing solutions A and B in a specific ratio for use. The division of the solutions into A and B prevents the reduction of its efficacy and efficiency that may occur in a mixed formulation. In addition, the solutions A and B have a different pH with each other and one of them contains a discoloring agent. The present first agent prepared by mixing the solutions A and B in a specific ratio for use shows a unique color caused by the color change agent (indicator, etc), which allows to identify its main ingredient and pH prior to operation. Therefore, a user can select the suitable first agent with referring to quality and condition of hair. Consequently, the first agent according to this invention ensures the prevention of hair damage, the securing of elastic wave and straight of hair and the shortening of the time of performance.

Accordingly, it is an object of this invention to provide a first agent for a permanent hair waving agent and a straightener.

### DISCLOSURE OF INVENTION

In one aspect of this invention, there is provided a first agent for a permanent hair waving agent and a hair straightener, wherein said first agent is divided into solution A and solution B, wherein each of the solutions A and B comprises a reducing agent, an alkali agent, a thickener, a humectant, a metal blocking agent, a hair tonic, a penetrant, a surfactant, a colorant, an emulsifier and a perfume, and the solution A, the solution B or both comprises 0.000001-10 wt % of a discoloring agent.

In another aspect of this invention, there is provided a first agent for a permanent hair waving agent and a hair straightener prepared by the method comprising the steps of: (i) dividing the first agent into solution A and solution B, wherein each of the solutions A and B comprises an alkali agent, a thickener, a humectant, a metal blocking agent, a hair tonic, a penetrant, a surfactant, a colorant, an emulsifier and a perfume; (ii) introducing a reducing agent into the solution A or solution B; and (iii) introducing a color change agent (indicator, etc) of 0.000001-10 wt % into the solution not containing the reducing agent, wherein the color change agent (indicator, etc) is selected from the group consisting of ninhydrin, sodium hydroxide, sodium nitrite and ferric chloride.

In still another aspect of this invention, there is provided a first agent for a permanent hair waving agent and a hair straightener prepared by the method comprising the steps of: (i) dividing the first agent into solution A and solution B, wherein each of the solutions A and B comprises an alkali agent, a thickener, a humectant, a metal blocking agent, a hair tonic, a penetrant, a surfactant, a colorant, an emulsifier and a perfume; (ii) introducing a reducing agent into the solution A or solution B; and (iii) introducing a metal ion of 0.001-1000 ppm into the solution not containing the reducing agent, wherein the metal ion is selected from the group consisting of Fe⁺⁺, Cu⁺⁺, Ag⁺⁺, Zn⁺⁺ and Pb⁺⁺.

In further aspect of this invention, there is provided a method of preparing a first agent for a permanent hair waving agent and a hair straightener, which comprises mixing the solutions A and B described above in a ratio of 0.1:99.9-99.9:0.1 to obtain the first agent having a pH of 4.5-9.6.

The present invention will be described in more detail as follows.

The present invention is directed to a first agent for a permanent hair waving agent and a hair straightener, prepared in such a manner that the first agent (i.e., a reducing agent) comprising thioglycolic acid or its salt and ester, cysteine and its salt and acetyl derivative as main ingredients, an alkali agent, a thickener, a humectant, a metal blocking agent, a hair tonic, a penetrant, a surfactant, a colorant, an emulsifier, a perfume, a reducing catalyst and a color change agent (indicator, etc) is divided into solution A and solution B. The solutions A and B are different with each other in view of their ingredients. When applied to, the solutions A and B are mixed to give a first agent with a specific color depending on ingredients and pH.

In general, a permanent hair waving agent and a hair straightener consist of a fist agent (i.e., reducing agent) containing a reducing agent, an alkali agent, a thickener, a penetrant, a stabilizer, a wetting agent, a hair tonic, an emulsifier, a colorant, a perfume and a metal blocking agent; and a second agent (i.e., an oxidizing agent) containing an oxidizing agent, a thickener, a stabilizer, a pH modifier and the like.

The present invention is characterized by a first agent between two agents of a permanent hair waving agent and a hair straightener.

In particular, the present invention relates to a first agent for a permanent hair waving agent and a hair straightener wherein the first agent is divided into solution A and solution B, where each of the solutions of A and B comprises a reducing agent, an alkali agent, a thickener, a humectant, a metal blocking agent, a hair tonic, a penetrant, a surfactant, a colorant, an emulsifier and a perfume, and the solution of (A), the solution of (B) or both comprises 0.000001-10 wt % of a color change agent (indicator, etc). Prior to operation, the solutions A and B are mixed at a predetermined ratio and then the color change agent (indicator, etc) contained in the solution A and/or (B) is color-developed. This color development enables the user to predetermine the property of the first agent (e.g. pH) and thus enables to select an appropriate permanent hair waving agent and a straightener according to the quality and condition of hair. As a result, the first agent according to this invention ensures the prevention of hair damage and the maintenance of elastic wave and straight configuration of hair. The amount of the color change agent (indicator, etc) in the first agent is in the range of 0.000001-10 wt %, preferably, 0.0001-5 wt %. If the amount is less than 0.000001 wt %, the discoloration in formulation is negligible; in case of exceeding 10 wt %, the extent of discoloration is excessive and the appearance and stability of the first agent becomes very poor. The reducing agent, alkali agent, thickener, humectant, metal blocking agent, hair tonic, penetrant, surfactant, colorant, emulsifier and perfume are conventionally available for preparing a permanent hair waving agent and a hair straightener. The ingredients described above are employed for enhancing the efficacy and efficiency of permanent hair waving agent and straightener.

Generally, when the pH of the first agent for a permanent hair waving agent and a straightener is in an alkaline range, it is applicable to oily, virgin, healthy and standard hairs. When the pH of the first agent is in a neutral range, it is applicable to standard, damaged and porous hairs. In addition, when the pH of the first agent is in an acidic range, it is suitable for porous, fine and severely damaged hairs. Therefore, when using the first agent of this invention, the pH of the first agent can be easily identified and thus can safely perform wave and straight hair styling according to the quality and condition of hair.

The examples of discoloring agents for identifying the pH of the first agent are shown in Table 2 and other conventional discoloring agents can be also used.

TABLE 2

| | | pH at | | | |
|---|---|---|---|---|---|
| | | color | Color in | Color in | |
| | | change | acid | alkali | |
| NO | Type | point, | solution | solution | Range |
| 1 | Methyl violet | 1.00 | Yellow | Blue | Acid |
| 2 | Dimethy yellow | 3.28 | Bluish | Green | |
| | | | purple | | |
| 3 | Methylene blue | | | | |
| 4 | Methyl yellow | 2.9-4.0 | Red | Yellow | |
| 5 | Bromophenol blue | 3.0-4.6 | Yellow | Blue | |
| 6 | Methyl orange | 4.10 | Purple | Green | |
| 7 | Indigocarmine | | | | |
| 8 | Hexamethoxytriphenyl | 4.00 | Purple | Green | |
| | Carbinol | | | | |
| 9 | Methyl green | | | | |
| 10 | Aniline blue | 4.30 | Purple | Green | |
| 11 | Methly red | 5.10 | Red | Green | |
| 12 | Bromocresol green | 6.10 | Yellowish | Bluish | |
| | | | green | purple | |
| 13 | Sodium alizarine | | | | |
| | Sulphonate | | | | |
| 14 | Bromphenol purple | 5.2-6.8 | Yellow | Green | |
| 15 | paranitrophenol | 7.0-7.5 | No color | Yellow | Neutral |
| 16 | Chlorophenol red | 5.80 | Green | Purple | |
| 17 | Bromthymol puple | 6.70 | Blue | Bluish | |
| | | | | purple | |
| 18 | Bromthymol blue | 6.80 | Yellow | Blue | |
| 19 | Azolitmin | 6.90 | Purple | Blue | |
| 20 | Neutral red | 7.00 | Bluish | Green | |
| | | | purple | | |
| 21 | Litmus | 7.2-8.8 | Red | Blue | |
| 22 | Cyanine | 7.30 | Yellow | Purple | |
| 23 | Phenol red | 7.30 | Yellow | Purple | |
| 24 | Cresol red | 7.2-8.8 | Yellow | Red | |
| 25 | Thymol blue | 8.30 | Yellow | Purple | Alkali |
| 26 | α-Na | 8.30 | Dark pink | Pink | |
| | phenolphthaleine | | | | |
| 27 | phenolphthaleine | 9.90 | No color | Pink | |
| 28 | α-Naphthalein | 9.60 | Dark pink | Pink | |
| 29 | Thymolphthaleine | 9.3-10.5 | No color | Blue | |
| 30 | Nile blue | 10.0 | Blue | Red | |
| 31 | Alizarine yellow | 10.20 | Yellow | Purple | |

Type of a mixed discoloring agent

| | | | | |
|---|---|---|---|---|
| 1 | methyl red + | 5.40 | Reddish | Blue |
| | Methylene blue | | purple | |
| 2 | Netual red + | 7.00 | Bluish | Blue |
| | Methylene blue | | purple | |
| 3 | Tymbollue + | 9.00 | Yellow | Purple |
| | Phenolphthalein | | | |

As indicated in Table 2, since the color change points (pH) are different from each other, the first agent prepared by mixing the solutions A and B ensures the predetermination of its liquid property (i.e., pH). For instance, the first agent obtained by mixing a colorless solution A having a pH of 3.2, where p-nitrophenol as a discoloring agent was introduced, with a colorless solution B having a pH of 9.5, reveals a pH of about 7.5 (neutrality) when measured after it turned to yellow. As such, because the property of the first agent is easily determined prior to a performance, it is possible to safely perform wave and straight hair styling according to the quality and condition of hair.

The present invention is directed to a first agent for a permanent hair waving agent and a hair straightener prepared by the method comprising the steps of: (i) dividing the first agent into solution A and solution B, wherein each of the solutions of A and B respectively comprises an alkali agent, a thickener, a humectant, a metal blocking agent, a hair tonic, a penetrant, a surfactant, a colorant, an emulsifier and a perfume; (ii) introducing a reducing agent into the solution A or solution B; and (iii) introducing a discoloring agent of 0.000001-10 wt % into the solution not containing the reducing agent, wherein the color change agent (indicator, etc) is selected from the group consisting of ninhydrin, sodium hydroxide, sodium nitrite and ferric chloride. The amount of the color change agent (indicator, etc) is in the range of 0.000001-10 wt %, preferably, 0.0001-5 wt %. If the amount is less than 0.000001 wt %, the color change in formulation is negligible; in case of exceeding 10 wt %, the extent of discoloration is excessive and the appearance and stability of the first agent becomes very poor. Since the color change agent (indicator, etc) containing ninhydrin, sodium hydroxide, sodium nitrite and ferric chloride can identify the main ingredients of the first agent, i.e. thioglycolic acids (thioglycolic acid, its salt and ester) and cysteines (cysteine, its salt and acetyl derivative) as reducing agents, it should be introduced into the solution selected from the either the solution A or the solution B, which does not contain a reducing agent. Preferably, the solution A or solution B without the reducing agent further comprises 0.001-1000 ppm of at least one reducing catalyst selected from the group consisting of Fe⁺⁺, Cu⁺⁺, Ag⁺⁺, Zn⁺⁺ and Pb⁺⁺. The metal ions make it possible to shorten performance time by facilitating the reduction action of a reducing agent in formulation of the first agent. For example, if ferric chloride, zinc chloride, copper chloride, silver nitrate, ferrous sulfide, copper sulfide or lead acetate as a reducing catalyst is used, the reduction action of thioglycolic acid and cysteine as a reducing agent is elevated, which enables to shorten the standing time from 30-40 min to 15-20 min during the preformance. Meanwhile, agents such as a reducing agent, an alkali agent, a thickener, a humectant, a metal blocking agent, a hair tonic, a penetrant, a surfactant, a colorant, an emulsifier and a perfume which are conventionally used in preparing a permanent hair waving agent and a hair straightener can be used in the present invention. The ingredients described above are employed for enhancing the efficacy and efficiency of a permanent hair waving agent and a straightener.

Table 3 shows the type of the discoloring agents to identify a reducing agent.

TABLE 3

| Color change | | |
|---|---|---|
| agent(indicator, | Color after mixing | Reducing agent |
| etc) | solutions A and B | (main ingredient) |
| Ninhydrin | Blue | Cysteines |
| Sodium hydroxide | Reddish purple | Cysteines |
| Sodium nitrite | Red | Thioglycolic acids |
| Ferric chloride | Red | Thioglycolic acids |

As indicated in Table 3, if the first agent containing ninhydrin and sodium hydroxide as a color change agent (indicator, etc) shows no color, it is determined to contain thioglycolic acids; and if it shows a blue or reddish purple color, it is determined to contain cysteines. Furthermore, if the first agent containing sodium nitrite and ferric chloride as a discoloring agent shows no color, it is determined to contain cysteines; and it shows a red color, it is determined to contain thioglycolic acids.

Generally, a first agent containing thioglycolic acids as a main ingredient is desirable for healthy, oily and standard hairs; and one containing cysteines as a main ingredient is appropriate for standard, porous, damaged and fine hairs. Therefore, when using the first agent of this invention that allows to predetermine a main ingredient, it is possible to perform wave and straight styling with safety according to the quality and condition of hair.

As described previously, the solutions A and B with a color change agent (indicator, etc) having pH 2.0-10.5 ix mixed at a certain ratio, preferably, 0.1:99.9-99.9:0.1 to obtain the first agent having pH of 4.5-9.6. At this time, the alteration of appearance (color) of the resulting first agent permits the user to determine the property and main ingredient of the first agent prior to operation. Therefore, the user can select the suitable first agent and thus the safety of the first agent is significantly increased.

### BRIEF DESCRIPTION OF DRAWINGS

FIG. 1 schematically shows the action mechanism of a permanent waving agent.

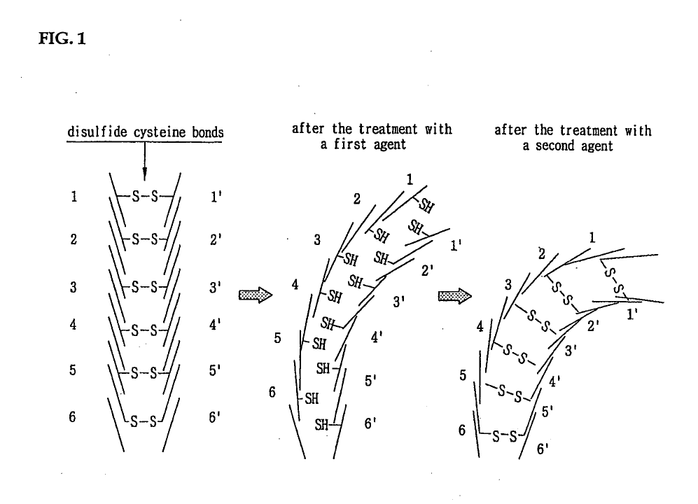

FIG. 2 schematically shows the action mechanism of a straightener.

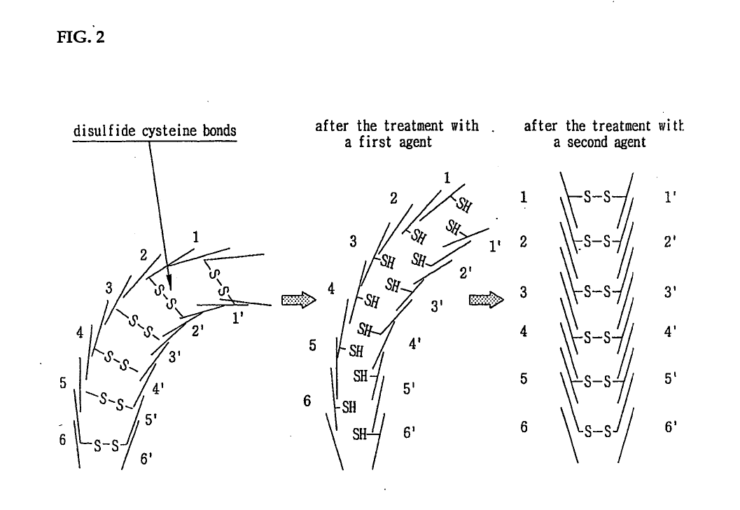

FIG. 3 schematically shows the action mechanism of both permanent hair waving agent and straightener.

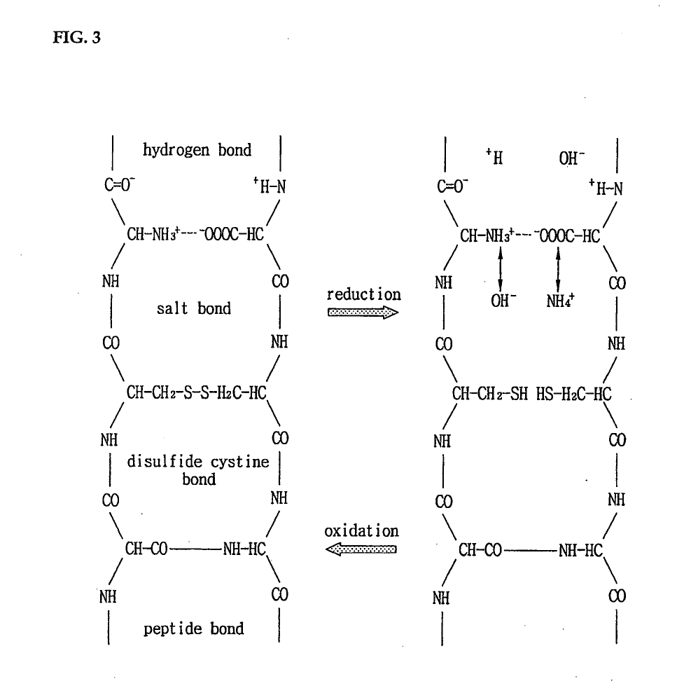

### BEST MODE FOR CARRYING OUT THE INVENTION

The following specific examples are intended to be illustrative of the invention and should not be construed as limiting the scope of the invention as defined by appended claims.

### EXAMPLES 1-3

The solution A of the first agent for a permanent hair waving agent and a hair straightener, which comprises the ingredients set forth in Table 4, was prepared in accordance with the conventional procedures for the first agent for a permanent hair waving agent and a hair straightener.

TABLE 4

| Ingredients (g) | | Ex. 1 | Ex. 2 | Ex. 3 |
|---|---|---|---|---|
| Hydroxyethylcellulose | | 0.50 | 0.50 | 0.50 |
| Glycerine | | 5.00 | 5.00 | 5.00 |
| Propylene glycol | | 2.00 | 2.00 | 2.00 |
| Etidronic acid (g) | | 0.10 | 0.10 | 0.10 |
| Main ingredient | L-cysteine | 0.00 | 8.00 | 0.00 |
| (reducing agent) | hydrochloride | | | |
| | monohydrate | | | |
| | Thioglycolic | 0.00 | 2.00 | 14.00 |
| | acid (50%) | | | |
| Urea | | 5.00 | 5.00 | 5.00 |
| Monoethanol amine | | 0.00 | 4.50 | 3.50 |
| polysorbate (20) | | 0.50 | 0.50 | 0.50 |
| Phosphoric acid | | 0.50 | — | — |
| Discoloring | p-nitrophenol | 1.50 | — | — |
| agent | (1.0%) | | | |
| | α- | — | 0.50 | — |
| | naphthalein | | | |
| | (1.0%) | | | |
| | Bromthymol | — | — | — |
| | blue (1.0%) | | | |

| | | | |
|---|---|---|---|
| Perfume | Adequate | Adequate | Adequate |
| | amount | amount | amount |

| | |
|---|---|
| Distilled water | To 100 g |

p-nitrophenol 97% (Research Chemicals LTD)
α-naphthalein (Special Grade) (Samchun Pure Chemical)
Bromthymol Blue (Special Grade) (Samchun Pure Chemical)

The solution B of the first agent for a permanent hair waving agent and a hair straightener, which comprises the ingredients set forth in Table 4, was prepared in accordance with the conventional procedures for the first agent for a permanent hair waving agent and a hair straightener.

| Ingredients (g) | | Ex. 1 | Ex. 2 | Ex. 3 |
|---|---|---|---|---|
| Hydroxyethylcellulose | | 0.50 | 0.50 | 0.50 |
| Glycerine | | 5.00 | 5.00 | 5.00 |
| Propylene glycol | | 2.00 | 2.00 | 2.00 |
| Etidronic acid (g) | | 0.10 | 0.10 | 0.10 |
| Main ingredient | L-cysteine | 8.00 | 1.00 | 3.00 |
| (reducing agent) | hydrochloride | | | |
| | monohydrate | | | |
| | Thioglycolic acid | 2.00 | 2.00 | 0.00 |
| | (50%) | | | |
| Urea | | 5.00 | 5.00 | 5.00 |
| Monoethanol amine | | 4.00 | 0.50 | 0.50 |
| Polysorbate (20) | | 0.50 | 0.50 | 0.50 |
| Discoloring | p-nitrophenol | — | — | — |
| agent | (1.0%) | | | |
| | A- | — | — | — |
| | naphthalein | | | |
| | (1.0%) | | | |
| | Bromthymol | — | — | 1.00- |
| | blue (1.0%) | | | |

| | | | |
|---|---|---|---|
| Perfume | Adequate | Adequate | Adequate |
| | amount | amount | amount |

| | |
|---|---|
| Distilled water | To 100 g |

p-nitrophenol 97% (Research Chemicals LTD)
α-naphthalein (Special Grade) (Samchun Pure Chemical)
Bromthymol Blue (Special Grade) (Samchun Pure Chemical)

### EXPERIMENTAL EXAMPLE

The solutions of A and B the first agent for a permanent hair waving agent and a hair straightener prepared in Examples 1-3 were mixed at the ratio indicated in Table 6. pH and color of mixtures were observed and summarized in Table 6.

TABLE 6

| | | pH | | | Color | | |
|---|---|---|---|---|---|---|---|
| | Mixing | Sol. | | First | | | First |
| | Ratio* | A | Sol. B | agent | Sol. A | Sol. B | agent |
| Ex. 1 | 1:4 | 3.20 | 9.50 | 7.50 | No color | No color | Yellow |
| Ex. 2 | 2:1 | 10.20 | 4.50 | 8.00 | Pink | No color | Dark |
| | | | | | | | pink |
| Ex. 3 | 3:1 | 9.30 | 5.00 | 9.00 | No color | Yellow | Blue |

*the ratio of sol. A to sol. B

As indicated in Table 6, the first agent for a permanent hair waving agent and a hair straightener according to the present invention is prepared by mixing the solutions A and B in a specific ratio that have different pH with each other. Thus, the color of the first agent is changed after formulation. Such a feature of the first agent permits the end user to select the first agent with a specific pH according to the quality and condition of hair, which results in the improvement in safety of a permanent hair waving agent and a hair straightener.

### EXAMPLE 4

The solution A of the first agent for a permanent hair waving agent and a hair straightener was prepared using the following ingredients in accordance with the conventional procedures.

To L-cysteine hydrochloride monohydrate (8.0 g), monoethanol amine (7.0 g), finecol-O (2.0 g), polypeptide (2.0 g) and finecol-EMS (20%) (10.0 g), distilled water was added to a final weight of 100 g.

The solution B of the first agent for a permanent hair waving agent and a hair straightener was prepared using the following ingredients in accordance with the conventional procedures.

To propylene glycol (2.0 g), glycerine (2.0 g), cetostearyl alcohol (5.0 g) and ninhydrin reagent (0.5 g), distilled water was added to a final weight of 100 g. Ninhydrin reagent was prepared by adding water to ninhydrin (99%) (Lancaster) at a final volume of 100 ml.

### EXPERIMENTAL EXAMPLE

The mixing of 90 g of the solution A and 10 g of the solution B resulted in the color development of bluish purple and thus it can be recognized that its main ingredient is cysteine or its salt. In addition, the color developed allows the user to understand the facts that the first agent can be safely applied to damaged hair, and a little heat or a longer time of performance is required when applied to healthy hair.

### EXAMPLE 5

The solution A of the first agent for a permanent hair waving agent and a hair straightener was prepared using the following ingredients in accordance with the conventional procedures.

To thioglycolic acid (50%) (14.0 g), ammonia water (25%) (2.0 g), finecol-O (2.0 g) and finecol-EMS (20%) (20.0 g), distilled water was added to a final weight of 100 g.

The solution B of the first agent for a permanent hair waving agent and a hair straightener was prepared using the following ingredients in accordance with the conventional procedures.

To polypeptide (3.0 g), propylene glycol (2.0 g) and ferric chloride (0.5 g, Kamto Chemical Co.), distilled water was added to a final weight of 100 g.

### EXPERIMENTAL EXAMPLE

The mixing of 80 g of the solution A and 20 g of the solution B of Example 5 resulted in the color development of red and thus it can be recognized that its main ingredient is thioglycolic acid, its salt or ester. In addition, the red color developed allows the user to understand the facts that the first agent is particularly suitable for standard, healthy and virgin hairs and Fe⁺⁺ serving as a catalyst for reduction by thioglycolic acid permits the shortening the time of performance.

### INDUSTRIAL APPLICABILITY

As described previously, the first agent for a permanent hair waving agent and a hair straightener according to the present invention is prepared by the separate solutions (A) and (B), which in turn are mixed in a specific ratio to use. The first agent permits the user to determine its property and main ingredient prior to operation. Therefore, the user can select the suitable first agent with referring to quality and condition of hair. Consequently, the first agent according to this invention ensures the prevention of hair damage, the securing of elastic wave and straight and the shortening of the time of performance.

What is claimed is:

1. A first agent for a permanent hair waving agent and a hair straightener, wherein said first agent is divided into solution A and solution B, wherein each of said solutions of A and B comprises a reducing agent, an alkali agent, a thickener, a humectant, a metal blocking agent, a hair tonic, a penetrant, a surfactant, a colorant, an emulsifier and a perfume, and said solution A, said solution B or both comprises 0.000001-10 wt % of a color change agent (indicator, etc).

2. The first agent according to claim 1, wherein said color change agent (indicator, etc) is at least one selected from the group consisting of Dimethyl yellow, Methylene blue, Methyl orange, Indigocarmine, Hexamethoxytriphenyl Carbinol, Methyl green, Aniline blue, Bromecresol green, Methyl red, Sodium alizarine sulphonate, Chlorophenol red, Bromthymol purple, Bromthymol blue, Azolitmin, Neutral red, Cyanine, Phenol, Phenol red, Cresol red, Thymol blue, α-Na phenolphthaleine, phenolphthaleine, α-Na phthalein, Thymolthaleine, Nile blue, Alizarine yellow, Methyl violet, Methyl yellow, Bromphenol blue, paranitrophenol, Bromphenol purple and Litmus.

3. The first agent according to claim 1, where the pH of said solution A is 2.0-10.5, the pH of said solution B is 2.0-10.5, and the pH of said first agent prepared by mixing said solutions A and B is 4.5-9.6.

4. A method of preparing a first agent for a permanent hair waving agent and a hair straightener, wherein said solutions A and B of claim 1 are mixed in a ratio of 0.1:99.9-99.9:0.1 to obtain said first agent having a pH of 4.5-9.6.

5. A first agent for a permanent hair waving agent and a hair straightener prepared by the method comprising the steps of: (i) dividing said first agent into solution A and solution B, wherein each of said solutions of A and B comprises an alkali agent, a thickener, a humectant, a metal blocking agent, a hair tonic, a penetrant, a surfactant, a colorant, an emulsifier and a perfume; (ii) introducing a reducing agent into said solution A or said solution B; and (iii) introducing a color change agent (indicator, etc) of 0.000001-10 wt % into the solution not containing said reducing agent, wherein said color change agent (indicator, etc) is selected from the group consisting of ninhydrin, sodium hydroxide, sodium nitrite and ferric chloride.

6. The first agent according to claim 5, wherein said solution A or B not containing said reducing agent further comprises 0.001-1000 ppm of at least one reducing catalyst selected from the group consisting of Fe⁺⁺, Cu⁺⁺, Ag⁺⁺, Zn⁺⁺ and Pb⁺⁺.

7. The first agent according to claim 5, wherein the pH of said solution A is 2.0-10.5, the pH of said solution B is 2.0-10.5, the pH of said first agent prepared by mixing said solutions A and B is 4.5-9.6.

8. A method of preparing a first agent for a permanent hair waving agent and a hair straightener, which comprises mixing said solutions A and B of claim 5 in a ratio of 0.1:99.9-99.9:0.1 to obtain said first agent having a pH of 4.5-9.6.
